SEQUENCE LISTING (18 sequences)

SEQ ID NO: 1 - protein, 338 aa, unidentified
MQLVDRVRGAVTGMSRRLVVGAVGAALVSGLVGAVGGTATAGAFSRPGLPVEYLQVPSPSMGRDIKVQFQSGGANSPALYLLDGLRAQDDFSGWDINTPAFEWYDQSGLSVVMPVGGQSSFYSDHWYQPACGKAGCQTYKWETFLTSELPGWLQANRHVKPTGSAVVGLSMAASSALTLAIYPQQFVYAGAMSGLLDPSQAMGPTLIGLAMGDAGGYKASDMWGPKEDPAWQRNDPLLNVGKLIANNTRVWVYCGNGKPSDLGGNNLPAKFLEGFVRTSNIKFQDAYNAGGGHNGVFDFPDSGTHSWEYWGAQLNAMKPDLQRALGATPNTGPAPQGA

SEQ ID NO: 2 - DNA, 1017 bp, unidentified
  source 1..1017: mol_type = unassigned DNA
atgcagcttgttgacagggttcgtggcgccgtcacgggtatgtcgcgtcgactcgtggtcggggccgtcggcgcggccctagtgtcgggtctggtcggcgccgtcggtggcacggcgaccgcgggggcattttcccggccgggcttgccggtggagtacctgcaggtgccgtcgccgtcgatgggccgtgacatcaaggtccaattccaaagtggtggtgccaactcgcccgccctgtacctgctcgacggcctgcgcgcgcaggacgacttcagcggctgggacatcaacaccccggcgttcgagtggtacgaccagtcgggcctgtcggtggtcatgccggtgggtggccagtcaagcttctactccgactggtaccagcccgcctgcggcaaggccggttgccagacttacaagtgggagaccttcctgaccagcgagctgccggggtggctgcaggccaacaggcacgtcaagcccaccggaagcgccgtcgtcggtctttcgatggctgcttcttcggcgctgacgctggcgatctatcacccccagcagttcgtctacgcgggagcgatgtcgggcctgttggacccctcccaggcgatgggtcccaccctgatcggcctggcgatgggtgacgctggcggctacaaggcctccgacatgtggggcccgaaggaggacccggcgtggcagcgcaacgacccgctgttgaacgtcgggaagctgatcgccaacaacacccgcgtctgggtgtactgcggcaacggcaagccgtcggatctgggtggcaacaacctgccggccaagttcctcgagggcttcgtgcggaccagcaacatcaagttccaagacgcctacaacgccggtggcggccacaacggcgtgttcgacttcccggacagcggtacgcacagctgggagtactggggcgcgcagctcaacgctatgaagcccgacctgcaacgggcactgggtgccacgcccaacaccgggcccgcgccccagggcgcctag

SEQ ID NO: 3 - protein, 299 aa, unidentified
MLRGIQALSRPLTRVYRALAVIGVLAASLLASWVGAVPQVGLAASALPTFAHVVIVVEENRSQAAIIGNKSAPFINSLAANGAMMAQAFAETHPSEPNYLALFAGNTFGLTKNTCPVNGGALPNLGSELLSAGYTFMGFAEDLPAVGSTVCSAGKYARKHVPWVNFSNVPTTLSVPFSAFPKPQNYPGLPTVSFVIPNADNDMHDGSIAQGDAWLNRHLSAYANWAKTNNSLLVVTWDEDDGSSRNQIPTVFYGAHVRPGTYNETISHYNVLSTLEQIYGLPKTGYATNAPPITDIWGD

SEQ ID NO: 4 - DNA, 900 bp, unidentified
  source 1..900: mol_type = unassigned DNA
atgctccgcggaatccaggctctcagccggcccctgaccagggtataccgtgccttggcggtgatcggtgtcctggcagcatcgttgctggcctcatgggtcggcgctgtcccacaagtgggtctggcagcgagtgccctgccgaccttcgcgcacgtggtcatcgtggtggaggagaaccgctcgcaggccgccatcatcggtaacaagtcggctcccttcatcaattcgctggccgccaacggcgcgatgatggcccaggcgttcgccgaaacacacccgagcgaaccgaactacctggcactgttcgctggcaacacattcgggttgacgaagaacacctgccccgtcaacggcggcgcgctgcccaacctgggttctgagttgctcagcgccggttacacattcatggggttcgccgaagacttgcctgcggtcggctccacggtgtgcagtgcgggcaaatacgcacgcaaacacgtgccgtgggtcaacttcagtaacgtgccgacgacactgtcggtgccgttttcggcatttccgaagccgcagaattaccccggcctgccgacggtgtcgtttgtcatccctaacgccgacaacgacatgcacgacggctcgatcgcccaaggcgacgcctggctgaaccgccacctgtcggcatatgccaactgggccaagacaaacaacagcctgctcgttgtgacctgggacgaagacgacggcagcagccgcaatcagatcccgacggtgttctacggcgcgcacgtgcggcccggaacttacaacgagaccatcagccactacaacgtgctgtccacattggagcagatctacggactgcccaagacgggttatgcgaccaatgctccgccaataaccgatatttggggcgactag

SEQ ID NO: 5 - protein, 663 aa, unidentified
VTLAIPSGIDLSHIDADARPQDDLFGHVNGRWLAEHEIPADRATDGAFRSLFDRAETQVRDLIIQASQAGAAVGTDAQRIGDLYASFLDEEAVERAGVQPLHDELATIDSAADATELAAALGTLQRAGVGGGIGVYVDTDSKDSTRYLVHFTQSGIGLPDESYYRDEQHAAVLAAYPGHIARMFGLVYGGESRDHAKTADRIVALETKLADAHWDVVKRRDADLGYNLRTFAQLQTEGAGFDWVSWVTALGSAPDAMTELVVRQPDYLVTFASLWASVNVEDWKCWARWRLIRARAPWLTRALVAEDFEFYGRTLTGAQQLRDRWKRGVSLVENLMGDAVGKLYVQRHFPPDAKSRIDTLVDNLQEAYRISISELDWMTPQTRQRALAKLNKFTAKVGYPIKWRDYSKLAIDRDDLYGNVQRGYAVNHDRELAKLFGPVDRDEWFMTPQTVNAYYNPGMNEIVFPAAILQPPFFDPQADEAANYGGIGAVIGHEIGHGFDDQGAKYDGDGNLVDWWTDDDRTEFAARTKALIEQYHAYTPRDLVDHPGPPHVQGAFTIGENIGDLGGLSIALLAYQLSLNGNPAPVIDGLTGMQRVFFGWAQIWRTKSRAAEAIRRLAVDPHSPPEFRCNGVVRNVDAFYQAFDVTEDDALFLDPQRRVRIWN

SEQ ID NO: 6 - DNA, 1992 bp, unidentified
  source 1..1992: mol_type = unassigned DNA
gtgacacttgccatcccctcgggtatcgacctgagccacatcgacgctgatgcccgaccccaagacgacctgttcggccacgttaacggccgctggctggctgaacacgagataccagcggaccgagcgaccgacggcgccttccgtagcctgttcgaccgcgccgagacacaagtgcgagacctgatcatccaggccagccaagcaggtgctgcggtaggcaccgatgcgcagcgcatcggcgacctctacgccagcttcctcgacgaggaagccgtcgagcgcgcaggggtgcaaccgctgcacgacgaattggccacgattgacagcgcggccgacgccaccgaattggccgccgcccttggcactctgcaacgtgccggcgtgggcggcggcatcggagtctatgtcgataccgattccaaagactcgacccgttacttggtgcatttcacccaatccggcatcggattacccgacgagtcctactaccgtgacgagcaacacgccgccgtgctagcggcctacccggggcacatcgcccggatgttcggcctggtgtacgggggcgagagccgtgaccatgccaaaaccgcggaccgcatcgtcgcgctggagaccaaactcgccgacgcgcattgggatgtggtgaagcgccgcgacgccgaccttggctacaacctgcgcacgtttgcccagctgcagaccgaaggggcgggtttcgactgggtcagctgggtgaccgcattggggagcgctccggacgccatgacggaactggttgtgcgccaacctgattacctcgtcacctttgcctcgctgtgggcgagcgttaacgttgaagactggaaatgctgggcgcgttggcgtttgatccgcgcccgggccccctggctgacccgcgccctggtcgccgaggacttcgaattctacggccgcacgcttaccggcgcacagcagcttcgggaccgttggaagcgtggggtgtcactggtggagaacctgatgggcgatgccgtcggaaagctctatgtacaacgccatttcccgccggatgccaagtcccgcatcgacaccctggtggacaacctgcaggaggcgtatcggatcagcatcagcgagctggattggatgacgccgcagacccggcaacgcgcgctagcgaagctgaacaagttcaccgccaaagtcggctatccgatcaagtggcgcgactactcgaagctggcgatcgaccgcgacgacctctacggtaacgtccagcgcggctacgccgtcaaccatgaccgcgagctagccaagcttttcggcccggtcgaccgcgacgagtggttcatgacaccacaaaccgtcaacgcctactacaacccggggatgaacgaaatcgtcttccccgcagcgattttacagccaccatttttcgatccgcaggccgacgaggccgccaactacggcgggatcggggcggtgatcgggcacgagatcgggcacggtttcgacgatcagggcgccaaatacgacggcgacggcaatctggtcgattggtggaccgacgacgatcgcaccgagttcgccgcccgcaccaaagcgttgatcgagcagtaccacgcttacacgccgcgcgatctcgtcgaccaccccggcccgcctcatgtgcaaggcgcgttcaccataggcgagaacatcggcgacctgggcgggctgtcgatcgccctgctggcttaccagctctcgctgaacggcaaccccgctccggttatcgacgggctgaccggcatgcaacgggtgttcttcggctgggcacaaatatggcgaaccaaatcgcgtgcagccgaagcaatccgccggttggcggtcgatccgcactccccgccggagttccggtgcaacggtgtggttcgcaacgtggacgctttttatcaggccttcgacgtcaccgaggatgacgcgctgtttctggacccgcagcgcagggtccggatctggaactag

SEQ ID NO: 7 - protein, 216 aa, unidentified
VVKVFLVDDEVVRRGLVDLLGADPELDVVGEAGSVAEAMARVPAARPDVAVLDVRLPDGNGIELCRDLLSRMPDLRCLILTSYTSDEAMLDAILAGASGYVVKDIKGMELARAVKDVGAGRSLLDNRAAAALMAKLRGAAEKQDPLSGLTDQERTLLGLLSEGLTNKQIADRMFLAEKTVKNYVSRLLAKLGMERRTQAAVFATELKRSRPPGDGP

SEQ ID NO: 8 - DNA, 654 bp, unidentified
  source 1..654: mol_type = unassigned DNA
gtggtaaaggtcttcttggtcgatgaccacgaggtggtgcgtcgtggtctggttgacttgcttggggccgatcccgagcttgacgtcgtaggtgaggcgggttcggtcgccgaggcgatggccagggttcctgccgcgcgcccagatgtcgcggtgctggatgtccggttgcccgatggcaacggcattgaactgtgccgcgatctgttgtcccgcatgcccgatctgcgctgtctgatcctcacgtcctacacctctgacgaggccatgctagatgcgattctcgccggtgccagcggatatgtcgtcaaagacatcaagggaatggagttggcgcgcgccgtcaaagatgtgggcgctggacggtcgctgctggacaatcgggccgcggccgcgctgatggccaagctgcgcggtgccgccgagaagcaggacccgctatcaggccttaccgaccaggagcggacgctactgggcctgcttagcgagggcctgaccaacaagcagatcgccgaccgaatgttcctagccgaaaagacggtgaagaactacgtgtcgcggttgctggccaagctgggcatggaacgtcggacgcaagccgcggtattcgcgacggagttgaagcgctcgcggccacccggtgatggaccatga

SEQ ID NO: 9 - protein, 583 aa, unidentified
MPVTDRSVPSLLQERADQQPDSTAYTYIDYGSDPKGFADSLTWSQVYSRACIIAEELKLCGLPGDRVAVLAPQGLEYVLAFLGALQAGFIAVPLSTPQYGIHDDRVSAVLQDSKPVAILTTSSVVGDVTKYAASHDGQPAPVVVEVDLLDLDSPRQMPAFSRQHTGAAYLQYTSGSTRTPAGVIVSHTNVIANVTQSMYGYFGDPAKIPTGTVVSWLPLYHDMGLILGICAPLVARRRAMLMSPMSFLRRPARWMQLLATSGRCFSAAPNFAFELAVRRTSDQDMAGLDLRDVVGIVSGSERIHVATVRRFIERFAPYNLSPTAIRPSYGLAEATLYVAAPEAGAAPKTVRFDYEQLTAGQARPCGTDGSVGTELISYGSPDPSSVRIVNPETMVENPPGVVGEIWVHGDHVTMGYWQKPKQTAQVFDAKLVDPAPAAPEGPWLRTGDLGVISDGELFIMGRIKDLLIVDGRNHYPDDIEATIQEITGGRAAAIAVPDDITEQLVAIIEFKRRGSTAEEVMLKLRSVKREVTSAISKSHSLRVADLVLVSPGSIPITTSGKIRRSACVERYRSDGFKRLDVAV

SEQ ID NO: 10 - DNA, 1752 bp, unidentified
  source 1..1752: mol_type = unassigned DNA
atgccggtgaccgaccgttcagtgccctctttgctgcaagagagggccgaccagcagcctgacagcactgcatatacgtacatcgactacggatccgaccccaagggatttgctgacagcttgacttggtcgcaggtctacagtcgtgcatgcatcattgctgaagaactcaagttatgcgggttacccggagatcgagtggcggttttagcgccacaaggactggaatatgtccttgcattcctgggcgcacttcaggctggatttatcgcggttccgctgtcaactccacagtatggcattcacgatgaccgcgtttctgcggtgttgcaggattccaagccggtagccattctcacgacttcgtccgtggtaggcgatgtaacgaaatacgcagccagccacgacgggcagcctgccccggtcgtagttgaggttgatctgcttgatttggactcgccgcgacagatgccggctttctctcgtcagcacaccggggcggcttatctccaatacacgtccggatcgacgcgtacgccggccggagtcattgtgtcgcacacgaatgtcattgccaatgtgacacaaagtatgtacggctatttcggcgatcccgcaaagattccgaccgggactgtggtgtcgtggctgcctttgtatcacgatatgggcctgattctcggaatttgcgcaccgctggtggcccgacgccgcgcgatgttgatgagcccaatgtcatttttgcgccgtccggcccgctggatgcaactgcttgccaccagcggccggtgcttttctgcggcaccgaatttcgccttcgagctggccgtgcgcagaacatctgaccaggacatggcggggctcgacctgcgcgacgtggtcggcatcgtcagtggcagtgagcgaatccatgtggcaaccgtgcggcggttcatcgagcggttcgcgccgtacaatctcagccccaccgcgatacggccgtcgtacgggctcgcggaagcgaccttatatgtggcagctcccgaagccggcgccgcgcccaagacggtccgttttgactacgagcagctgaccgccgggcaggctcggccctgcggaaccgatgggtcggtcggcaccgaactgatcagctacggctcccccgacccatcgtctgtgcgaatcgtcaacccggagaccatggttgagaatccgcctggagtggtcggtgagatctgggtgcatggcgaccacgtgactatggggtattggcagaagccgaagcagaccgcgcaggtcttcgacgccaagctggtcgatcccgcgccggcagccccggaggggccgtggctgcgcaccggcgacctgggcgtcatttccgatggtgagctgttcatcatgggccgcatcaaagacctgctcatcgtggacgggcgcaaccactaccccgacgacatcgaggcaacgatccaggagatcaccggtggacgggccgcggcgatcgcagtgcccgacgacatcaccgaacaactggtggcgatcatcgaattcaagcgacgcggtagtaccgccgaagaggtcatgctcaagctccgctcggtgaagcgtgaggtcacctccgcgatatcgaagtcacacagcctgcgggtggccgatctcgttctggtgtcacctggttcgattcccatcaccaccagcggcaagatccggcggtcagcctgcgtcgaacgctatcgcagcgacggcttcaagcggctggacgtagccgtatga

SEQ ID NO: 11 - protein, 216 aa, unidentified
MADIDGVTGSAGLQPGPSEETDEELTARFERDAIPLLDQLYGGALRMTRNPADAEDLLQETMVKAYAGFRSFRHGTNLKAWLYRILTNTYINSYRKKQRQPAEYPTEQITDWQLASNAEHSSTGLRSAEVEALEALPDTEIKEALQALPEEFRMAVYYADVEGFPYKEIAEIMDTPIGTVMSRLHRGRRQLRGLLADVARDRGFARGEQAHEGVSS

SEQ ID NO: 12 - DNA, 651 bp, unidentified
  source 1..651: mol_type = unassigned DNA
atggccgacatcgatggtgtaaccggttcggcgggtctgcagcctgggccgtctgaggagacagacgaggagttgaccgcgcgtttcgagcgcgacgcgattcccctgttggaccagctgtacggcggtgcgctgcggatgacgcgcaatccggccgacgccgaggacttgctccaggagacgatggtgaaggcctatgcgggatttcgttcgttccggcacggtaccaatctcaaggcctggctctaccggatactgaccaacacctacatcaacagctatcgcaagaaacagcggcaaccggcggagtatccgaccgagcagatcaccgattggcaactggcgtccaacgccgagcattcctcgaccgggctgcgctcggctgaagtcgaagcgttagaagcgttgccggacaccgagatcaaagaggcgctgcaggcattgccggaagagttccggatggcggtctactacgccgatgtcgaaggtttcccctacaaggagatcgccgagatcatggatactccgatcggcaccgtgatgtcgaggcttcatcgcggccgacgtcagttgcgcggtcttttagccgatgtggccagggatcgggggtttgccaggggcgagcaggcgcacgagggggtgtcgtcatga

SEQ ID NO: 13 - protein, 806 aa, unidentified
VTQAADTDIRVGQPEMVTLTIDGVEISVPKGTLVIRAAELMGIQIPRFCDHPLLEPVGACRQCLVEVEGQRKPLASCTTVATDDMVVRTQLTSEIADKAQHGVMELLLINHPLDCPMCDKGGECPLQNQAMSNGRTDSRFTEAKRTFAKPINISAQVLLDRERCILCARCTRFSDQIAGDPFIDMQERGALQQVGIYADEPFESYFSGNTVQICPVGALTGTAYRFRARPFDLVSSPSVCEHCASGCAQRTDHRRGKVLRRLAGDDPEVNEEWNCDKGRWAFTYATQPDVITTPLIRDGGDPKGALVPTSWSHAMAVAAQGLAAARGRTGVLVGGRVTWEDAYAYAKFARITLGTNDIDFRARPHSAEEADFLAARIAGRHMAVSYADLESAPVVLLVGFEPEDESPIVFLRLRKAARRHRVPVYTIAPFATGGLHKMSGRLIKTVPGGEPAALDDLATGAVGDLLATPGAVIIVGERLATVPGGLSAAARLADTTGARLAWVPRRAGERGALEAGALPTLLPGGRPLADEVARAQVCAAWHIAELPAAAGRDADGILAAAADETLAALLVGGIEPADFADPDAVLAALDATGFVVSLELRHSTVTERADVVFPVAPTTQKAGAFVNWEGRYRTFEPALRGSTLQAGQSDHRVLDALADDMGVHLGVPTVEAAREELAALGIWDGKHAAGPHIAATGPTQPEAGEAILTGWRMLLDEGRLQDGEPYLAGTARTPVVRLSPDTAAEIGAADGEAVTVSTSRGSITLPCSVTDMPDRVVWLPLNSAGSTVHRQLRVTIGSIVKIGAGS

SEQ ID NO: 14 - DNA, 2421 bp, unidentified
  source 1..2421: mol_type = unassigned DNA
gtgacccaggcggccgacactgacatccgggtaggccaaccggagatggtgacactgaccatcgacggcgtcgaaatcagcgtccccaagggcacgttggtgattcgcgccgccgaactgatgggaatccagatcccgcgattctgcgaccacccgctgctggagcccgtcggcgcctgccggcaatgcctggtcgaggtcgaagggcaacgcaagccgctggcgtcgtgcaccaccgtggccaccgacgacatggtggtgcgcacccaactcacctccgagattgccgacaaggcccagcacggtgtgatggaactgctgctgatcaaccatccgctggattgcccgatgtgcgacaagggcggtgaatgcccgctgcaaaaccaggcaatgtctaacggccgcacggattctcgcttcaccgaggccaaacgtaccttcgccaaaccgatcaacatctccgcgcaggtgctgctggaccgcgaacgttgcatcctgtgcgcccgctgcacccggttctccgaccagatcgccggcgatccgttcatcgatatgcaggagcgcggcgccctgcagcaggtcggtatctacgccgatgaaccgttcgagtcgtacttctccggcaacacggtgcagatctgcccggtgggggcgctaacggggaccgcctaccggttccgcgcgcgtccgttcgatttggtctccagccccagcgtctgcgagcactgcgcgtcgggctgcgcgcaacgcaccgaccatcgccgcggcaaggtgctgcggcggctggccggtgacgacccggaagtcaacgaggagtggaactgcgacaagggccggtgggccttcacgtacgcgacccagccggacgtgatcaccactcccctgatccgcgacggtggggaccccaagggcgcgctggtgcccacctcgtggtcgcacgcaatggcggtggccgcccagggactggcggcagcgcggggccgcaccggggtgctggtcggcggccgagtgacctgggaggacgcctacgcgtacgccaagttcgcgcggatcacgttgggcaccaacgacatcgacttccgcgcccggccgcactcggccgaggaggccgacttcctggcggcccgcatcgccgggcggcatatggcggtcagctatgccgatttggaatcggctccggtggtgctgctggtgggattcgagcccgaagacgagtcgccgatcgtgtttctgcggttacgcaaggccgctcgcagacaccgcgtcccggtgtacacgatcgccccctttgccactggtggcctgcacaaaatgtcgggccggctgatcaaaaccgttcctggtggcgaacccgcggcgctggacgatctggccaccggtgcagtgggcgacctgctggccaccccgggcgcggtcatcatagtcggggagcgcttggccacggtaccgggcggattgtcggcggccgctcggctggccgatacgaccggcgcccgtttggcgtgggtgccgcggcgggcgggggaacgcggagcgctggaagccggagcgttgcccacgctgttacccggtggccgcccgctggccgacgaggtcgcccgcgcgcaggtgtgtgcggcgtggcatatcgccgaattgcctgccgcggctggacgggacgccgacggcatcctggccgccgctgccgacgagacgttggctgcgctgctggtcgggggtatcgaacccgcggacttcgccgacccggacgccgtgctggccgcgttggacgccaccggtttcgtggtcagcctggagctgcgacacagtacggtcaccgaacgcgccgacgtggtgttcccggtcgcgccgacgacccagaaagccggcgcgttcgtcaactgggagggtcgctaccgtacattcgaacccgcgctgcgcggcagcacactgcaagctggccagtcggatcaccgggtgctggacgcgttggccgacgacatgggtgtccatctgggcgtgcccaccgtggaggcggcccgcgaggagctggccgcgctcggtatctgggacggcaaacacgctgccggtccccacatcgcggccaccgggccgacccaacccgaagctggtgaggcgatcttgaccgggtggcggatgctcctcgacgagggccgcctgcaggacggcgaaccatatctggccggtaccgcgcgcacacccgtggtacggctgtcgccggatacggcagccgagatcggcgccgccgatggcgaggcggtcacggtcagcacgtcacgcggctcaatcaccttgccgtgcagtgtcaccgacatgcccgaccgcgtcgtgtggcttccgctgaactcggcgggctcgacggtgcaccgacagctgagggtgacaatcggcagcatcgtgaaaatcggagcgggctcatga

SEQ ID NO: 15 - protein, 402 aa, unidentified
VTVTLCSPTEDDWPGMFLLAAASFTDFIGPESATAWRTLVPTDGAVVVRDGAGPGSEVVGMALYMDLRLTVPGEVVLPTAGLSFVAVAPTHRRRGLLRAMCAELHRRIADSGYPVAALHASEGGIYGRFGYGPATTLHELTVDRRFARFHADAPGGGLGGSSVRLVRPTEHRGEFEAIYERWRQQVPGGLLRPQVLWDELLAECKAAPGGDRESFALLHPDGYALYRVDRTDLKLARVSELRAVTADAHCALWRALIGLDSMERISIITHPQDPLPHLLTDTRLARTTWRQDGLWLRIMNVPAALEARGYAHEVGEFSTVLEVSDGGRFALKIGDGRARCTPTDAAAEIEMDRDVLGSLYLGAHRASTLAAANRLRTKDSQLLRRLDAAFASDVPVQTAFEF

SEQ ID NO: 16 - DNA, 1209 bp, unidentified
  source 1..1209: mol_type = unassigned DNA
gtgactgtgaccctgtgtagcccgaccgaggacgactggccggggatgttcctactggccgcggccagtttcaccgatttcatcggccctgaatcagcgaccgcctggcggaccctggtgcccaccgacggagcggtggtggtccgcgatggtgccggcccgggttctgaggtggtcgggatggcgctgtacatggatctgcggttgacggtgcctggtgaagtggtgctcccgaccgccggtctcagtttcgtcgcggtggcgccgacgcatcgccggcgcggcttgctgcgcgcgatgtgcgccgaactgcaccgccgcatagccgattccggctatccggtcgcggcactgcatgctagcgagggcggcatctacggccggttcggctacgggcccgctaccaccttgcatgagctgacggtcgaccgacgcttcgcgcgctttcacgccgacgcaccgggcggcggcctaggtggcagcagcgtccggttggtcagacccaccgagcatcgcggcgagtttgaggcgatctacgagcgatggcgccagcaggtgccgggcgggctgctacgcccgcaggtgctctgggacgagctgctggcagaatgcaaagccgcgcccggtggagaccgtgaatcgttcgcgttactgcatcccgacgggtacgcgctgtaccgggtggatcgcaccgatctcaagctagcgcgcgtcagcgaactcagggcggtaaccgcagatgcgcattgtgcgttgtggcgggccctgattggcctcgactccatggagcgaatcagcatcatcacccatccacaggacccgttaccccacctgctcaccgatacccgactggcccgcactacctggcgccaggacggcctgtggttgcgcatcatgaacgtaccggccgcactcgaggcgcgtggttacgctcacgaagttggcgagttttccacggtcctcgaggtatccgatggcggccggttcgcgctcaagatcggtgacggccgtgcgcggtgtaccccgaccgatgcggcagccgagatcgaaatggatcgggacgtactgggcagcctttaccttggagcgcaccgcgcttcgacgttagccgccgctaaccggttgcgcaccaaagattcccagctgcttcgtcgactcgacgcggcgtttgccagtgatgttcccgtccagaccgcgttcgagttctga

SEQ ID NO: 17 - protein, 95 aa, unidentified
MTEQQWNFAGIEAAASAIQGNVTSIHSLLDEGKQSLTKLAAAWGGSGSEAYQGVQQKWDATATELNNALQNLARTISEAGQAMASTEGNVTGMFA

SEQ ID NO: 18 - protein, 100 aa, unidentified
MAEMKTDAATLAQEAGNFERISGDLKTQIDQVESTAGSLQGQWRGAAGTAAQAAVVRFQEAANKQKQELDEISTNIRQAGVQYSRADEEQQQALSSQMGF

(19) United States
(12) Patent Application Publication
(10) Pub. No.: US 2025/0332240 A1
(43) Pub. Date: Oct. 30, 2025

(54) NOVEL VACCINES FOR TUBERCULOSIS

(71) Applicants: TEXAS TECH UNIVERSITY SYSTEM, Lubbock, TX (US); BOARD OF REGENTS OF THE UNIVERSITY OF TEXAS SYSTEM, Austin, TX (US); THE METHODIST HOSPITAL, Houston, TX (US)

(72) Inventors: Subramanian Dhandayuthapani, Edinburg,, TX (US); Kishore Das, Austin, TX (US); Omar A. Garnica, El Paso,, TX (US); Chinnaswamy Jagannath, Sugar Land,, TX (US); Raja Veerapandian, El Paso,, TX (US)

(73) Assignees: TEXAS TECH UNIVERSITY SYSTEM, Lubbock, TX (US); BOARD OF REGENTS OF THE UNIVERSITY OF TEXAS SYSTEM, Austin, TX (US); THE METHODIST HOSPITAL, Houston, TX (US)

(21) Appl. No.: 18/869,804

(22) PCT Filed: May 26, 2023
(86) PCT No.: PCT/US2023/023677

§ 371 Date: Nov. 26, 2024

Related U.S. Application Data
(60) Provisional application No. 63/346,576, filed on May 27, 2022.

(51) Int. Cl. A61K 39/04; A61K 39/00; A61P 31/06; C12N 1/20; C12R 1/32

(52) U.S. Cl. CPC A61K 39/04; A61P 31/06; C12N 1/20; A61K 2039/522; A61K 2039/542; A61K 2039/543; A61K 2039/572; C12R 2001/32

(57) ABSTRACT

Embodiments of the present disclosure pertains to a genetically altered bacterial strain that lacks functional versions of at least three of the following proteins: FbpA; SapM; Zmp1; DosR; FadD26; SigH; nuoG; and Eis. In some embodiments, the bacterial strain lacks functional versions of at least the following proteins: FbpA; SapM; Zmp1; and DosR. In some embodiments, the bacterial strain lacks functional versions of at least the following proteins: FbpA; SapM; Zmp1; DosR; FadD26; and SigH. In some embodiments, the bacterial strain lacks functional versions of at least the following proteins: SapM; Zmp1; and nuoG. Further embodiments of the present disclosure pertain to methods of treating or preventing a bacterial infection in a subject by administering to the subject a bacterial strain of the present disclosure. In some embodiments, the bacterial infection is tuberculosis.

### CROSS-REFERENCE TO RELATED APPLICATIONS

This application claims priority to U.S. Provisional Patent Application No. 63/346,576, filed on May 27, 2022. The entirety of the aforementioned application is incorporated herein by reference.

### STATEMENT REGARIDNG FEDERALLY SPONSORED RESEARCH

This invention was made with government support under 1R01AI175837-01 awarded by the National Institutes of Health. The government has certain rights in the invention.

### STATEMENT UNDER 37 C.F.R. $1.834 (C) (1)

Pursuant to 37 C.F.R. § 1.834, Applicant hereby submits a sequence listing as an XML file (“Sequence Listing”). The name of the file containing the Sequence Listing is “AF13368.P039WO.xml”. The date of the creation of the Sequence Listing is May 26, 2023. The size of the Sequence Listing is 30,000 bytes. Applicant hereby incorporates by reference the material in the Sequence Listing.

### BACKGROUND

Current vaccines for treating or preventing bacterial infections have numerous limitations. Embodiments of the present disclosure aim to address the aforementioned limitations.

### SUMMARY

In some embodiments, the present disclosure pertains to a genetically altered bacterial strain. In some embodiments, the bacterial strain lacks functional versions of at least three of the following proteins: FbpA; SapM; Zmp1; DosR; FadD26; SigH; nuoG; and Eis.

In some embodiments, the bacterial strain lacks functional versions of at least the following proteins: FbpA; SapM; Zmp1; and DosR. In some embodiments, the bacterial strain also lacks a functional version of the FadD26 protein. In some embodiments, the bacterial strain also lacks a functional version of the SigH protein. In some embodiments, the bacterial strain lacks functional versions of at least the following proteins: FbpA; SapM; Zmp1; DosR; FadD26; and SigH. In some embodiments, the bacterial strain lacks functional versions of at least the following proteins: SapM; Zmp1; and nuoG. In some embodiments, the bacterial strain lacks functional versions of at least the following proteins: SapM; Zmp1; and Eis. In some embodiments, the bacterial strain lacks functional versions of at least the following proteins: SapM; Zmp1; Eis; and nuoG.

Further embodiments of the present disclosure pertain to methods of treating or preventing a bacterial infection in a subject by administering to the subject a bacterial strain of the present disclosure. In some embodiments, the bacterial infection is tuberculosis.

### DESCRIPTION OF THE DRAWINGS

FIG. 1 provides data indicating that a Mycobacterium tuberculosis (Mtb) ΔfbpA-ΔsapM double knockout strain (DKO) vaccine shows improved protection against Mtb infection in a mouse model. C57BL/6 mice immunized with DKO and BCG strains were challenged with virulent Mtb after one-month post-immunization. The challenged mice were sacrificed after 30 days and the amount of bacteria in the lungs were enumerated by plating the extracts of the lung in mycobacterial agar growth medium (7H11). Results show that the Mtb growth in DKO immunized mice was significantly lower (1 log₁₀) compared to BCG immunized mice.

FIGS. 2A-2C provide polymerase chain reaction (PCR) confirmation for the deletion of genes in TKO-Z, TKO-D and QKO1 strains. To confirm the deletion of sapM, dosR and zmp1 genes in TKO-Z, TKO-D and QKO1 strains, PCR was conducted using gene specific primers (primers Rv3310EX1 and Rv3310EX2 for sapM; primers DOSR5 and DOSR6 for dosR gene; primers ZMP5 and ZMP6 for zmp1) and genomic DNA templates from all three mutated strains and from the wild type H37Rv. The PCR results revealed appropriate gene deletion by amplifying lower size DNA fragments in the mutants than the wild type H37Rv, confirming deletions.

FIG. 3 shows in vitro immunogenicity by vaccine strains. Mouse bone marrow derived macrophages (BMDMs) from naïve C57BL/6 mice were infected with M. tuberculosis strains (MOI=1:5) for 4 h and cocultured with BB7 T cell hybridoma specific for Ag85B₂₄₁₋₂₅₆ peptide. After 16 h, culture fluids were collected and assayed for IL-2 levels released by the BB7 cells in response to Ag85B peptide. Culture fluids from BMDMs infected with QKO1 strain and cocultured with BB7 T cells showed significantly elevated levels of IL-2 as compared to BMDMs infected with other strains (p values<0.05 were considered statistically significant).

FIGS. 4A-4F provide data indicating that the QKO1 vaccine strain show enhanced lysosomal localization in primary mouse macrophages. BMDMs from naïve C57BL/6 mice were infected with rfpH37Rv (FIG. 4A), rfpDKO (FIG. 4B), rfpTKO-D (FIG. 4C), rfpTKO-Z (FIG. 4D), and rfpQKO1 (FIG. 4E) (MOI=1:1) for 4 h, washed, incubated for 24 hrs and stained with primary antibodies to lysosomal marker Rab7 followed by staining with FITC (green) conjugated secondary antibody. Mycobacteria colocalizing with antibodies were scored using a Nikon TiE Fluorescence Microscope and Metaview Deconvolution Software. Illustrations indicate that the TKO-Z and QKO1 mutants colocalize better with Rab7 lysosomal marker. FIG. 4F shows percent colocalization was determined by counting 50 macrophages per well each with 1-3 mycobacteria and averaging counts from triplicate slides (SD). One of three similar experiments is shown (p values<0.05 were considered statistically significant).

FIGS. 5A-5F provide data indicating that the TKO-Z and QKO1 vaccine strains exhibit increased autophagy induction in primary mouse macrophages. BMDMs from naïve C57BL/6 mice were infected with rfpH37Rv (FIG. 5A), rfpDKO (FIG. 5B), rfpTKO-D (FIG. 5C), rfpTKO-Z (FIG. 5D), and rfpQKO1 (FIG. 5E) (MOI=1:1) for 4 h, washed, incubated for 24 hrs and stained with primary antibodies to autophagosomal marker LC3 followed by staining with FITC (green) conjugated secondary antibody. Mycobacteria colocalizing with antibodies were scored using a Nikon TiE Fluorescence Microscope and Metaview Deconvolution Software. Illustrations indicate that the TKO-Z and QKO1 mutants co-localize better with LC3 marker. FIG. 5F shows that percent colocalization was determined by counting 50 macrophages per well each with 1-3 mycobacteria and averaging counts from triplicate chambers (SD). One of three similar experiments is shown (p values<0.05 were considered statistically significant).

FIGS. 6A-6G show induction of apoptosis by vaccine strains. BMDMs isolated from naïve C57BL/6 mice were infected with Mtb strains: PBS control (FIG. 6A), H37Rv (FIG. 6B), DKO (FIG. 6C), TKO-Z (FIG. 6D), TKO-D (FIG. 6E), and QKO1 (FIG. 6F) for 4 hrs after which they were washed and further incubated for another 12-18 hrs. Cells were then incubated with FITC Annexin V in a buffer containing propidium iodide (PI) and analyzed by flow cytometry using a BD FACS Canto II instrument. As summarized in FIG. 6G, the four genetically altered vaccine strains (DKO, TKO-D, TKO-Z and QKO1) induced significantly higher amounts of apoptotic cells when compared to wild-type H37Rv. P values<0.05 were considered statistically significant.

FIGS. 7A-7C show data indicating that QKO1 antigens are processed by all three mechanisms. BMDMs from naïve C57BL/6 mice were pretreated with phagolysosomal pathway inhibitors (cathepsin B (CatBi) or bafilomycin (Bafilo)), apoptosis inhibitors (z-VAD or z-VAD) or autophagolysosome pathway inhibitor (3-methyl adenine (3MA)) and infected with M. tuberculosis strains (MOI=1:5) H37Rv, DKO or QKO1 for 4 h. The infected cells were cocultured with BB7 T cell hybriodma specific for Ag85B₂₄₁₋₂₅₆ peptide. After 16 h, culture fluids were collected and assayed for IL-2 levels released by the BB7 cells in response to Ag85B peptide. Release of IL-2 levels by BB7 cells cocultured with BMDMs infected with QKO1 were greatly affected by all inhibitors, specifically by AP and apoptosis inhibitors. P values<0.05 were considered statistically significant.

FIGS. 8A-8E show in vivo immunogenicity by vaccine strains. C57BL/6 mice (both males and females) were vaccinated with BCG, DKO, TKO-D, TKO-Z, and QKO1 vaccine strains (1×10⁶ subcutaneously) and the control H37Rv. After 30 days post-immunization, mice were euthanized and spleens were isolated. Splenocytes (2.5×10⁵/well) were plated and stimulated in vitro for 48 h with Mtb H37Rv whole cell lysate (20 μg/ml). Supernatants from cultures were collected and, IFN-γ (FIG. 8A), IL-1β (FIG. 8B), IL-2 (FIG. 8C), IL-12 (FIG. 8D), and TNF (FIG. 8E) levels determined by ELISA. P values<0.05 were considered statistically significant.

FIG. 9 summarizes the profiles of IFN-γ producing splenocytes in vaccinated mice. C57BL/6 mice (of both sexes) were vaccinated with BCG, DKO, TKO-D, TKO-Z, and QKO1 vaccine strains and control H37Rv (1×10⁶ subcutaneously). After 30 days post-immunization, mice were euthanized and spleens were isolated. Splenocytes (2.5×10⁵/well) were plated and stimulated with a combination of Ag85B and CFP-10 peptides in vitro for 48 h. Ag85B/CFP-10 responsive IFN-γ producing spleens cells were spotted using IFN-γ ELISPOT plates following manufacturer's protocols. QKO1 vaccination leads to a larger expansion of Ag85B/CFP-10 specific T cells when compared to BCG, H37Rv, DKO and TKO-D. P values<0.05 were considered statistically significant.

FIG. 10 shows that the QKO1 vaccine strain is attenuated in macrophages. BMDMs (pre-activated with IFN-γ) from naïve C57BL/6 mice were infected with mycobacteria (MOI 1:1) for 4 h, washed, incubated, lysed and plated for viable colony counts (CFUs). The graph illustrates that the QKO1 strain is severely attenuated when compared to wild type Mtb (H37Rv), DKO, and TKO-Z. P values<0.05 were considered statistically significant.

### DETAILED DESCRIPTION

It is to be understood that both the foregoing general description and the following detailed description are illustrative and explanatory, and are not restrictive of the subject matter, as claimed. In this application, the use of the singular includes the plural, the word “a” or “an” means “at least one”, and the use of “or” means “and/or”, unless specifically stated otherwise. Furthermore, the use of the term “including”, as well as other forms, such as “includes” and “included”, is not limiting. Also, terms such as “element” or “component” encompass both elements or components comprising one unit and elements or components that include more than one unit unless specifically stated otherwise.

The section headings used herein are for organizational purposes and are not to be construed as limiting the subject matter described. All documents, or portions of documents, cited in this application, including, but not limited to, patents, patent applications, articles, books, and treatises, are hereby expressly incorporated herein by reference in their entirety for any purpose. In the event that one or more of the incorporated literature and similar materials defines a term in a manner that contradicts the definition of that term in this application, this application controls.

Bacterial infections present numerous global health issues. For instance, tuberculosis (TB), caused by the bacterial pathogen Mycobacterium tuberculosis (Mtb), is a global health issue and is a significant cause of disability and mortality throughout the world.

In 2018 alone, TB was responsible for approximately 1.45 million deaths. The World Health Organization (WHO) estimates that 10.0 million new cases of TB occur each year globally and one quarter of the world population carries latent TB infection (LTBI). Moreover, the emergence of multidrug-resistant tuberculosis (MDR-TB) and extensively drug resistant tuberculosis (XDR-TB) strains, and the infection of immunocompromised AIDS patients by Mtb, worsen this situation.

These complications lead to not only treatment and management issues but also financial burdens because treatment of MDR/XDR TB is very expensive. The WHO has proposed to eradicate TB by the year 2050 and meeting this objective requires intensive research towards the development of improved drugs and vaccines for the treatment and prevention of TB, respectively.

Moreover, vaccines for preventing bacterial infections have limited efficacies. For instance, Bacille Calmette-Guerin (BCG) remains the only approved vaccine against tuberculosis (TB). However, BCG's capability in preventing the progression of infection to disease has limitations. For instance, although BCG is considered safe and partially effective against extra-pulmonary childhood TB, its ability to protect against childhood and adult pulmonary TB is still questionable. In addition, there is a concern that BCG does not induce long lasting immune responses in the immunized individuals. Moreover, the efficacy of BCG varies drastically (e.g., 0-80%) between different ethnic populations and age groups.

BCG's safety profile has prompted the researchers to improve its efficacy by alternate methods. Most of these attempts have focused on altering its antigenic makeup through recombinant DNA technology. Examples of these second generation BCG vaccines include recombinant strains such as rBCG30, which overexpresses the immunodominant antigen Ag85B (8); BCG::RD1 vaccine, in which BCG was complemented with RD1 region antigens; and rBCG:ΔureC:hly (VPM1002), which expresses listeriolysin (LLO). Some of these BCG vaccines are now in phase-I or phase-II clinical trials.

In addition to improving the BCG vaccine, development of novel vaccine candidates from Mtb itself has been attempted in the past two decades not only to find a replacement for BCG but also to retain the full antigenic repertoire of Mtb. These include vaccines based on Mtb auxotrophs for purine, leucine, proline/tryptophan, lysine and pantothenate. The vaccines also include Mtb strains that carry deletions of specific genes, such as fadD26, mec-2/mec-3, ΔRD1/panCD, phoP, 19 kDa, sigE, fbpA, secA2/lysA, phoP/fadD26 (MTBVAC), phoP/fadD26/erp, sigH, mosR, echA7, and sigE/fadD26. Among these, the strains ΔRD1/panCD and MTBVAC are in phase I clinical trial, although both are defective in ESAT-6 and CFP-10 expression or secretion.

Similar to wild-type (parental) BCG, ΔRD1/panCD does not express ESAT-6 and CFP-10. Meanwhile, MTBVAC is defective in protein translocation of ESAT-6 and CFP-10 across the cell wall. As a result, these two Mtb based vaccine strains provide efficacy equivalent to or slightly better than that of non-recombinant BCG. However, none of the vaccines show complete protection against TB in mouse models, thereby necessitating the improvement of Mtb-derived vaccines.

In sum, a need exists for improved vaccines for preventing bacterial infections, such as TB. Numerous embodiments of the present disclosure aim to address the aforementioned need.

### Genetically Altered Bacterial Strains

In some embodiments, the present disclosure pertains to a genetically altered bacterial strain. In some embodiments, the bacterial strain lacks functional versions of at least three of the following proteins: FbpA; SapM; Zmp1; DosR; FadD26; SigH; nuoG, and Eis.

In some embodiments, the bacterial strain lacks functional versions of at least the following proteins: FbpA; SapM; Zmp1; and DosR. In some embodiments, the bacterial strain also lacks a functional version of the FadD26 protein. In some embodiments, the bacterial strain also lacks a functional version of the SigH protein. In some embodiments, the bacterial strain lacks functional versions of at least the following proteins: FbpA; SapM; Zmp1; DosR; FadD26; and SigH.

In some embodiments, the bacterial strain lacks functional versions of at least the following proteins: SapM; Zmp1; and nuoG. In some embodiments, the bacterial strain lacks functional versions of at least the following proteins: SapM; Zmp1; and Eis. In some embodiments, the bacterial strain lacks functional versions of at least the following proteins: SapM; Zmp1; Eis and nuoG. In some embodiments, the bacterial strain is in isolated form.

The bacterial strains of the present disclosure may lack functional versions of one or more of the aforementioned proteins in various manners. For instance, in some embodiments, the bacterial strains of the present disclosure may entirely lack one or more of the aforementioned proteins. In some embodiments, the bacterial strains of the present disclosure may include non-functional versions of one or more of the aforementioned proteins. In some embodiments, the bacterial strains of the present disclosure may include a mutant version of one or more of the aforementioned proteins. In some embodiments, a mutant version of a protein disrupts or eliminates a function of the protein. In some embodiments, the bacterial strains of the present disclosure may include a truncated version of one or more of the aforementioned proteins.

In some embodiments, the bacterial strains of the present disclosure lack a functional version of FbpA. In some embodiments, the FbpA protein includes SEQ ID NO: 1. In some embodiments, the FbpA protein includes a sequence with at least 65% sequence identity to SEQ ID NO: 1. In some embodiments, the FbpA protein includes a sequence with at least 70% sequence identity to SEQ ID NO: 1. In some embodiments, the FbpA protein includes a sequence with at least 75% sequence identity to SEQ ID NO: 1. In some embodiments, the FbpA protein includes a sequence with at least 80% sequence identity to SEQ ID NO: 1. In some embodiments, the FbpA protein includes a sequence with at least 85% sequence identity to SEQ ID NO: 1. In some embodiments, the FbpA protein includes a sequence with at least 90% sequence identity to SEQ ID NO: 1. In some embodiments, the FbpA protein includes a sequence with at least 95% sequence identity to SEQ ID NO: 1.

In some embodiments, the bacterial strains of the present disclosure include a mutation or deletion of fbpA, the gene for the FbpA protein. In some embodiments, fbpA includes SEQ ID NO: 2. In some embodiments, fbpA includes a sequence with at least 65% sequence identity to SEQ ID NO: 2. In some embodiments, fbpA includes a sequence with at least 70% sequence identity to SEQ ID NO: 2. In some embodiments, fbpA includes a sequence with at least 75% sequence identity to SEQ ID NO: 2. In some embodiments, fbpA includes a sequence with at least 80% sequence identity to SEQ ID NO: 2. In some embodiments, fbpA includes a sequence with at least 85% sequence identity to SEQ ID NO: 2. In some embodiments, fbpA includes a sequence with at least 90% sequence identity to SEQ ID NO: 2. In some embodiments, fbpA includes a sequence with at least 95% sequence identity to SEQ ID NO: 2.

In some embodiments, the bacterial strains of the present disclosure lack a functional version of SapM. In some embodiments, the SapM protein includes SEQ ID NO: 3. In some embodiments, the SapM protein includes a sequence with at least 65% sequence identity to SEQ ID NO: 3. In some embodiments, the SapM protein includes a sequence with at least 70% sequence identity to SEQ ID NO: 3. In some embodiments, the SapM protein includes a sequence with at least 75% sequence identity to SEQ ID NO: 3. In some embodiments, the SapM protein includes a sequence with at least 80% sequence identity to SEQ ID NO: 3. In some embodiments, the SapM protein includes a sequence with at least 85% sequence identity to SEQ ID NO: 3. In some embodiments, the SapM protein includes a sequence with at least 90% sequence identity to SEQ ID NO: 3. In some embodiments, the SapM protein includes a sequence with at least 95% sequence identity to SEQ ID NO: 3.

In some embodiments, the bacterial strains of the present disclosure include a mutation or deletion of sapM, the gene for the SapM protein. In some embodiments, sapM includes SEQ ID NO: 4. In some embodiments, sapM includes a sequence with at least 65% sequence identity to SEQ ID NO: 4. In some embodiments, sapM includes a sequence with at least 70% sequence identity to SEQ ID NO: 4. In some embodiments, sapM includes a sequence with at least 75% sequence identity to SEQ ID NO: 4. In some embodiments, sapM includes a sequence with at least 80% sequence identity to SEQ ID NO: 4. In some embodiments, sapM includes a sequence with at least 85% sequence identity to SEQ ID NO: 4. In some embodiments, sapM includes a sequence with at least 90% sequence identity to SEQ ID NO: 4. In some embodiments, sapM includes a sequence with at least 95% sequence identity to SEQ ID NO: 4.

In some embodiments, the bacterial strains of the present disclosure lack a functional version of Zmp1. In some embodiments, the Zmp1 includes SEQ ID NO: 5. In some embodiments, the Zmp1 protein includes a sequence with at least 65% sequence identity to SEQ ID NO: 5. In some embodiments, the Zmp1 protein includes a sequence with at least 70% sequence identity to SEQ ID NO: 5. In some embodiments, the Zmp1 protein includes a sequence with at least 75% sequence identity to SEQ ID NO: 5. In some embodiments, the Zmp1 protein includes a sequence with at least 80% sequence identity to SEQ ID NO: 5. In some embodiments, the Zmp1 protein includes a sequence with at least 85% sequence identity to SEQ ID NO: 5. In some embodiments, the Zmp1 protein includes a sequence with at least 90% sequence identity to SEQ ID NO: 5. In some embodiments, the Zmp1 protein includes a sequence with at least 95% sequence identity to SEQ ID NO: 5.

In some embodiments, the bacterial strains of the present disclosure include a mutation or deletion of zmp1, the gene for the Zmp1 protein. In some embodiments, zmp1 includes SEQ ID NO: 6. In some embodiments, zmp1 includes a sequence with at least 65% sequence identity to SEQ ID NO: 6. In some embodiments, zmp1 includes a sequence with at least 70% sequence identity to SEQ ID NO: 6. In some embodiments, zmp1 includes a sequence with at least 75% sequence identity to SEQ ID NO: 6. In some embodiments, zmp1 includes a sequence with at least 80% sequence identity to SEQ ID NO: 6. In some embodiments, zmp1 includes a sequence with at least 85% sequence identity to SEQ ID NO: 6. In some embodiments, zmp1 includes a sequence with at least 90% sequence identity to SEQ ID NO: 6. In some embodiments, zmp1 includes a sequence with at least 95% sequence identity to SEQ ID NO: 6.

In some embodiments, the bacterial strains of the present disclosure lack a functional version of DosR. In some embodiments, the DosR protein includes SEQ ID NO: 7. In some embodiments, the DosR protein includes a sequence with at least 65% sequence identity to SEQ ID NO: 7. In some embodiments, the DosR protein includes a sequence with at least 70% sequence identity to SEQ ID NO: 7. In some embodiments, the DosR protein includes a sequence with at least 75% sequence identity to SEQ ID NO: 7. In some embodiments, the DosR protein includes a sequence with at least 80% sequence identity to SEQ ID NO: 7. In some embodiments, the DosR protein includes a sequence with at least 85% sequence identity to SEQ ID NO: 7. In some embodiments, the DosR protein includes a sequence with at least 90% sequence identity to SEQ ID NO: 7. In some embodiments, the DosR protein includes a sequence with at least 95% sequence identity to SEQ ID NO: 7.

In some embodiments, the bacterial strains of the present disclosure include a mutation or deletion of dosR, the gene for the DosR protein. In some embodiments, dosR includes SEQ ID NO: 8. In some embodiments, dosR includes a sequence with at least 65% sequence identity to SEQ ID NO: 8. In some embodiments, dosR includes a sequence with at least 70% sequence identity to SEQ ID NO: 8. In some embodiments, dosR includes a sequence with at least 75% sequence identity to SEQ ID NO: 8. In some embodiments, dosR includes a sequence with at least 80% sequence identity to SEQ ID NO: 8. In some embodiments, dosR includes a sequence with at least 85% sequence identity to SEQ ID NO: 8. In some embodiments, dosR includes a sequence with at least 90% sequence identity to SEQ ID NO: 8. In some embodiments, dosR includes a sequence with at least 95% sequence identity to SEQ ID NO: 8.

In some embodiments, the bacterial strains of the present disclosure lack a functional version of FadD26. In some embodiments, the FadD26 protein includes SEQ ID NO: 9. In some embodiments, the FadD26 protein includes a sequence with at least 65% sequence identity to SEQ ID NO: 9. In some embodiments, the FadD26 protein includes a sequence with at least 70% sequence identity to SEQ ID NO: 9. In some embodiments, the FadD26 protein includes a sequence with at least 75% sequence identity to SEQ ID NO: 9. In some embodiments, the FadD26 protein includes a sequence with at least 80% sequence identity to SEQ ID NO: 9. In some embodiments, the FadD26 protein includes a sequence with at least 85% sequence identity to SEQ ID NO: 9. In some embodiments, the FadD26 protein includes a sequence with at least 90% sequence identity to SEQ ID NO: 9. In some embodiments, the FadD26 protein includes a sequence with at least 95% sequence identity to SEQ ID NO: 9.

In some embodiments, the bacterial strains of the present disclosure include a mutation or deletion of fadD26, the gene for the FadD26 protein. In some embodiments, fadD26 includes SEQ ID NO: 10. In some embodiments, fadD26 includes a sequence with at least 65% sequence identity to SEQ ID NO: 10. In some embodiments, fadD26 includes a sequence with at least 70% sequence identity to SEQ ID NO: 10. In some embodiments, fadD26 includes a sequence with at least 75% sequence identity to SEQ ID NO: 10. In some embodiments, fadD26 includes a sequence with at least 80% sequence identity to SEQ ID NO: 10. In some embodiments, fadD26 includes a sequence with at least 85% sequence identity to SEQ ID NO: 10. In some embodiments, fadD26 includes a sequence with at least 90% sequence identity to SEQ ID NO: 10. In some embodiments, fadD26 includes a sequence with at least 95% sequence identity to SEQ ID NO: 10.

In some embodiments, the bacterial strains of the present disclosure lack a functional version of SigH. In some embodiments, the SigH protein includes SEQ ID NO: 11. In some embodiments, the SigH protein includes a sequence with at least 65% sequence identity to SEQ ID NO: 11. In some embodiments, the SigH protein includes a sequence with at least 70% sequence identity to SEQ ID NO: 11. In some embodiments, the SigH protein includes a sequence with at least 75% sequence identity to SEQ ID NO: 11. In some embodiments, the SigH protein includes a sequence with at least 80% sequence identity to SEQ ID NO: 11. In some embodiments, the SigH protein includes a sequence with at least 85% sequence identity to SEQ ID NO: 11. In some embodiments, the SigH protein includes a sequence with at least 90% sequence identity to SEQ ID NO: 11. In some embodiments, the SigH protein includes a sequence with at least 95% sequence identity to SEQ ID NO: 11.

In some embodiments, the bacterial strains of the present disclosure include a mutation or deletion of sigH, the gene for the SigH protein. In some embodiments, sigH includes SEQ ID NO: 12. In some embodiments, sigH includes a sequence with at least 65% sequence identity to SEQ ID NO: 12. In some embodiments, sigH includes a sequence with at least 70% sequence identity to SEQ ID NO: 12. In some embodiments, sigH includes a sequence with at least 75% sequence identity to SEQ ID NO: 12. In some embodiments, sigH includes a sequence with at least 80% sequence identity to SEQ ID NO: 12. In some embodiments, sigH includes a sequence with at least 85% sequence identity to SEQ ID NO: 12. In some embodiments, sigH includes a sequence with at least 90% sequence identity to SEQ ID NO: 12. In some embodiments, sigH includes a sequence with at least 95% sequence identity to SEQ ID NO: 12.

In some embodiments, the bacterial strains of the present disclosure lack a functional version of nuoG. In some embodiments, the nuoG protein includes SEQ ID NO: 13. In some embodiments, the nuoG protein includes a sequence with at least 65% sequence identity to SEQ ID NO: 13. In some embodiments, the nuoG protein includes a sequence with at least 70% sequence identity to SEQ ID NO: 13. In some embodiments, the nuoG protein includes a sequence with at least 75% sequence identity to SEQ ID NO: 13. In some embodiments, the nuoG protein includes a sequence with at least 80% sequence identity to SEQ ID NO: 13. In some embodiments, the nuoG protein includes a sequence with at least 85% sequence identity to SEQ ID NO: 13. In some embodiments, the nuoG protein includes a sequence with at least 90% sequence identity to SEQ ID NO: 13. In some embodiments, the nuoG protein includes a sequence with at least 95% sequence identity to SEQ ID NO: 13.

In some embodiments, the bacterial strains of the present disclosure include a mutation or deletion of nuoG, the gene for the nuoG protein. In some embodiments, nuoG includes SEQ ID NO: 14. In some embodiments, nuoG includes a sequence with at least 65% sequence identity to SEQ ID NO: 14. In some embodiments, nuoG includes a sequence with at least 70% sequence identity to SEQ ID NO: 14. In some embodiments, nuoG includes a sequence with at least 75% sequence identity to SEQ ID NO: 14. In some embodiments, nuoG includes a sequence with at least 80% sequence identity to SEQ ID NO: 14. In some embodiments, nuoG includes a sequence with at least 85% sequence identity to SEQ ID NO: 14. In some embodiments, nuoG includes a sequence with at least 90% sequence identity to SEQ ID NO: 14. In some embodiments, nuoG includes a sequence with at least 95% sequence identity to SEQ ID NO: 14.

In some embodiments, the bacterial strains of the present disclosure lack a functional version of Eis. In some embodiments, the Eis protein includes SEQ ID NO: 15. In some embodiments, the Eis protein includes a sequence with at least 65% sequence identity to SEQ ID NO: 15. In some embodiments, the Eis protein includes a sequence with at least 70% sequence identity to SEQ ID NO: 15. In some embodiments, the Eis protein includes a sequence with at least 75% sequence identity to SEQ ID NO: 15. In some embodiments, the Eis protein includes a sequence with at least 80% sequence identity to SEQ ID NO: 15. In some embodiments, the Eis protein includes a sequence with at least 85% sequence identity to SEQ ID NO: 15. In some embodiments, the Eis protein includes a sequence with at least 90% sequence identity to SEQ ID NO: 15. In some embodiments, the Eis protein includes a sequence with at least 95% sequence identity to SEQ ID NO: 15.

In some embodiments, the bacterial strains of the present disclosure include a mutation or deletion of Eis, the gene for the Eis protein. In some embodiments, Eis includes SEQ ID NO: 16. In some embodiments, Eis includes a sequence with at least 65% sequence identity to SEQ ID NO: 16. In some embodiments, Eis includes a sequence with at least 70% sequence identity to SEQ ID NO: 16. In some embodiments, Eis includes a sequence with at least 75% sequence identity to SEQ ID NO: 16. In some embodiments, Eis includes a sequence with at least 80% sequence identity to SEQ ID NO: 16. In some embodiments, Eis includes a sequence with at least 85% sequence identity to SEQ ID NO: 16. In some embodiments, Eis includes a sequence with at least 90% sequence identity to SEQ ID NO: 16. In some embodiments, Eis includes a sequence with at least 95% sequence identity to SEQ ID NO: 16.

In some embodiments, the bacterial strains of the present disclosure include a functional version of ESAT6. In some embodiments, the ESAT6 protein includes SEQ ID NO: 17. In some embodiments, the ESAT6 protein includes a sequence with at least 65% sequence identity to SEQ ID NO: 17. In some embodiments, the ESAT6 protein includes a sequence with at least 70% sequence identity to SEQ ID NO: 17. In some embodiments, the ESAT6 protein includes a sequence with at least 75% sequence identity to SEQ ID NO: 17. In some embodiments, the ESAT6 protein includes a sequence with at least 80% sequence identity to SEQ ID NO: 17. In some embodiments, the ESAT6 protein includes a sequence with at least 85% sequence identity to SEQ ID NO: 17. In some embodiments, the ESAT6 protein includes a sequence with at least 90% sequence identity to SEQ ID NO: 17. In some embodiments, the ESAT6 protein includes a sequence with at least 95% sequence identity to SEQ ID NO: 17.

In some embodiments, the bacterial strains of the present disclosure include a functional version of CFP10. In some embodiments, the CFP10 protein includes SEQ ID NO: 18. In some embodiments, the CFP10 protein includes a sequence with at least 65% sequence identity to SEQ ID NO: 18. In some embodiments, the CFP10 protein includes a sequence with at least 70% sequence identity to SEQ ID NO: 18. In some embodiments, the CFP10 protein includes a sequence with at least 75% sequence identity to SEQ ID NO: 18. In some embodiments, the CFP10 protein includes a sequence with at least 80% sequence identity to SEQ ID NO: 18. In some embodiments, the CFP10 protein includes a sequence with at least 85% sequence identity to SEQ ID NO: 18. In some embodiments, the CFP10 protein includes a sequence with at least 90% sequence identity to SEQ ID NO: 18. In some embodiments, the CFP10 protein includes a sequence with at least 95% sequence identity to SEQ ID NO: 18.

The bacterial strains of the present disclosure may be derived from various bacterial species. For instance, in some embodiments, the bacterial strains of the present disclosure include Mycobacterium tuberculosis (Mtb). In some embodiments, the bacterial strains of the present disclosure include Mycobacterium bovis BCG (BCG).

### Compositions

In some embodiments, the bacterial strains of the present disclosure may be in a composition. Further embodiments of the present disclosure pertain to compositions that contain the bacterial strains of the present disclosure. In some embodiments, the compositions of the present disclosure may be suitable for administration to a subject. In some embodiments, the compositions of the present disclosure may be suitable for use in treating or preventing a bacterial infection in a subject.

In some embodiments, the compositions of the present disclosure may be formulated for administration in one or more doses. In some embodiments, the compositions of the present disclosure may be the form of a vaccine.

In some embodiments, the compositions of the present disclosure help make the bacterial strains of the present disclosure suitable for administration. In some embodiments, the compositions of the present disclosure also include one or stabilizers. In some embodiments, the stabilizers include, without limitation, anti-oxidants, sequestrants, ultraviolet stabilizers, or combinations thereof.

In some embodiments, the compositions of the present disclosure also include one or more surfactants. In some embodiments, the surfactants include, without limitation, anionic surfactants, sugars, cationic surfactants, zwitterionic surfactants, non-ionic surfactants, or combinations thereof.

In some embodiments, the compositions of the present disclosure also include one or more excipients. In some embodiments, the excipients include, without limitation, lactose, sucrose, starch powder, cellulose esters of alkanoic acids, trehalose, cellulose alkyl esters, talc, stearic acid, magnesium stearate, magnesium oxide, sodium and calcium salts of phosphoric and sulfuric acids, gelatin, acacia gum, trehalose, sodium alginate, polyvinylpyrrolidone, polyvinyl alcohol, or combinations thereof.

In some embodiments, the compositions of the present disclosure include a delivery vehicle, such as a particle. In some embodiments, the particle includes, without limitation, lipid-based particles, carbon-based particles, metal-based particles, or combinations thereof. In some embodiments, the active agents of the present disclosure are encapsulated in the particle.

### Methods of Treating or Preventing a Bacterial Infection

In some embodiments, the bacterial strains of the present disclosure are suitable for use in treating or preventing a bacterial infection in a subject. Further embodiments of the present disclosure pertain to methods of treating or preventing a bacterial infection in a subject by administering to the subject a bacterial strain of the present disclosure.

The methods of the present disclosure may be utilized to treat or prevent various bacterial infections. For instance, in some embodiments, the bacterial infection includes, without limitation, tuberculosis, leprosy, mycobacterial infections, bacterial infections associated with viral infections (e.g., SARS-CoV-2), or combinations thereof. In some embodiments, the bacterial infection is tuberculosis.

In some embodiments, the methods of the present disclosure may be utilized to prevent the bacterial infection. In some embodiments, the methods of the present disclosure may be utilized to mitigate the bacterial infection. In some embodiments, the methods of the present disclosure may be utilized to treat the bacterial infection.

The bacterial strains of the present disclosure may be administered to various subjects. For instance, in some embodiments, the subject is a human being. In some embodiments, the subject is a non-human animal. In some embodiments, the non-human animal includes, without limitation, a cat, a dog, a mouse, a cattle or a horse.

In some embodiments, the subject is vulnerable to a bacterial infection. In some embodiments, the subject is suffering from a bacterial infection.

The bacterial strains of the present disclosure may be administered to subjects in various manners. For instance, in some embodiments, the administering occurs by methods that include, without limitation, intravenous administration, subcutaneous administration, transdermal administration, topical administration, intraarterial administration, intrathecal administration, intracranial administration, intraperitoneal administration, intraspinal administration, intranasal administration, intraocular administration, oral administration, intratumor administration, and combinations thereof.

Without being bound by theory, the administered bacterial strains of the present disclosure may have various effects in subjects. For instance, in some embodiments, the administered bacterial strains elicit an enhanced immune response against the bacterial infection in the subject. In some embodiments, the enhanced immune response is characterized by enhanced phagolysosomal processing of the bacterial strain by antigen presenting cells when compared to genetically un-altered bacterial strains (e.g., bacterial strains that contain functional versions of at least one of FbpA, SapM, Zmp1, DosR, FadD26, SigH, nuoG; and Eis). In some embodiments, the enhanced immune response is characterized by enhanced IL-2 production in the subject when compared to IL-2 production from administered genetically un-altered bacterial strains. In some embodiments, the enhanced immune response is characterized by enhanced IL-1β production in the subject when compared to IL-1β production from administered genetically un-altered bacterial strains. In some embodiments, the enhanced immune response is characterized by enhanced IL-12 production in the subject when compared to IL-12 production from administered genetically un-altered bacterial strains. In some embodiments, the enhanced immune response is characterized by enhanced INF-γ production in the subject when compared to INF-γ production from administered genetically un-altered bacterial strains. In some embodiments, the enhanced immune response is characterized by enhanced TNF-α production in the subject when compared to TNF-α production from administered genetically un-altered bacterial strains.

### ADDITIONAL EMBODIMENTS

Reference will now be made to more specific embodiments of the present disclosure and experimental results that provide support for such embodiments. However, Applicants note that the disclosure below is for illustrative purposes only and is not intended to limit the scope of the claimed subject matter in any way.

### Example 1. Quadruple Knockout Bacterial Strains as a Vaccine Against Tuberculosis

To improve Mycobacterium tuberculosis (Mtb)-derived vaccines, Applicants developed the concept called ‘rationally deleting genes in Mtb genome’. This concept involved mainly targeting Mtb genes that play critical roles in immune evasion mechanisms or pathogenesis, particularly those associated with prevention of phagolysosomal (PL) fusion in antigen presenting cells (APCs) such as macrophages and dendritic cells. The rationale is that once the factors associated with the prevention of PL fusion is aborted, then the antigenic molecules of the vaccine will be fully processed by APCs, and presented to the host immune cells, which will eventually lead to increased efficacy of the vaccine. To demonstrate this concept, Applicants first selected the gene sapM of Mtb. This gene codes for an acid phosphatase, SapM, which is not only a secreted protein but also a protein that strongly interferes with the maturation of phagosomes or the prevention/evasion of PL fusion.

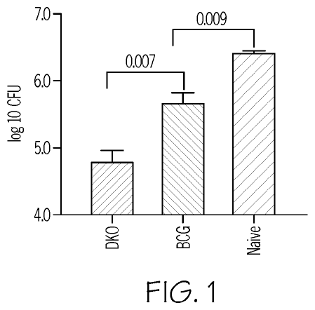

For deleting sapM, Applicants used an Mtb strain deficient in FbpA or Ag85A protein. Applicants preferred this strain over wild type Mtb because this mutant strain (ΔfbpA) has been shown to be relatively PL fusion competent and immunogenic as compared to wild type Mtb in mice. The resulting Mtb ΔfbpA-ΔsapM double knockout strain was named as DKO. The DKO strain was found to be efficiently processed through the PL pathway and APCs infected with this strain presented significantly higher levels of antigen than APCs infected with their parental strains. Further, the DKO strain is highly immunogenic. In fact, mice immunized with the DKO strain showed enhanced protection against TB compared to mice immunized with BCG (FIG. 1).

Similar to Applicants' observations, deletion of sapM in BCG has also been shown to improve its efficacy. These observations suggested that rational deletion of genes is the most appropriate approach to develop novel and efficacious Mtb-derived vaccines against TB. Towards this end, Applicants generated a Quadruple Knockout 1 (QKO1) strain by sequentially deleting zmp1 and dosR genes in the Mtb DKO vaccine strain. While zmp1 gene codes for a metalloprotease that affects inflammasome activation in the host cells, dosR gene codes for a transcriptional repressor associated with hypoxia survival. Both genes have been shown to have a role in the prevention of phagosomal maturation in APCs.

As observed in Applicants' in vitro studies, the QKO1 vaccine strain is efficiently processed by APCs through PL fusion, autophagy and apoptosis pathways leading to superior antigen presentation. Further, Applicants' in vivo studies in mouse indicate that QKO1 is markedly immunogenic and induce antigen specific Th1 responses. Thus, the scientific premise is that the high immunogenicity and strong attenuation of QKO1 will translate into enhanced efficacy and safety, and consequently QKO1 will become an effective Mtb-derived new generation vaccine against TB.

In this Example, Applicants assessed the protective efficacy as well as safety of QKO1 in a mouse model of infection. In particular, Applicants have generated an additional vaccine strain QKO1 (lacking in FbpA, SapM, Zmp1 and DosR proteins) by deleting the zmp1 and dosR genes in the DKO strain. Applicants' preliminary results indicate that this strain is not only processed by the PL fusion pathway more efficiently but also through alternate mechanisms such as autophagolysosome (APL) pathway and apoptosis, thus making this a highly immunogenic vaccine strain. In addition, unlike other Mtb-derived vaccine strains that are in clinical trials such as MtbΔRD1/panCD and MTBVAC, the QKO1 has intact genes for the expression of the ESAT6 and CFP10 immunogenic proteins.

### Example 1.1. Generation of QKO1 Vaccine Strain

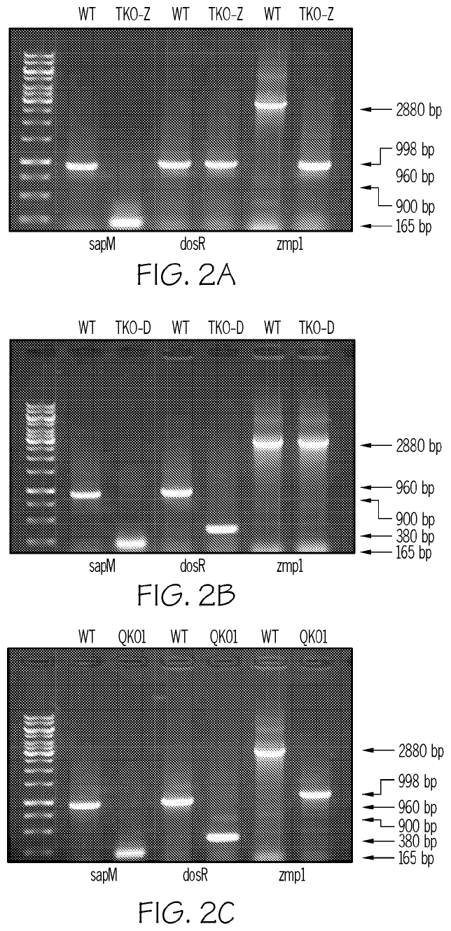

Applicants previously generated a DKO (ΔfbpA-ΔsapM) mutant strain through homologous recombination and assessed its efficacy (FIG. 1). Although the DKO strain exhibits higher efficacy than BCG, it still retains some virulence when tested in SCID mice, an immunocompromised mouse model traditionally used for assessing the safety aspect standpoint of mycobacterial vaccines. In order to reduce its virulence and simultaneously to improve its immunogenicity/efficacy, Applicants generated additional deletion of genes through homologous recombination. Applicants chose the genes zmp1 and dosR of Mtb, which encode a metalloprotease and a response regulator, respectively. The zmp1 and dosR genes were allelically replaced, without using antibiotic markers, by introducing the knockout plasmid constructs pKNOCK197 and pKNOCK3133, respectively. Initially, Applicants generated triple knockouts TKO-Z (ΔfbpA-ΔsapM-Δzmp1) and TKO-D (ΔfbpA-ΔsapM-ΔdosR) by introducing the plasmids pKNOCK197 and pKNOCK3133 separately into DKO strain. The QKO1 (ΔfbpA-ΔsapM-ΔdosR-Δzmp1) was then obtained by introducing the plasmid pKNOCK197 in the triple knockout strain TKO-D. The PCR data, based on gene specific primers, presented in FIGS. 2A-2C, confirmed the deletion of appropriate genes in the strains tested. Applicants have also confirmed these gene deletions in these strains by genome sequencing.

### Example 1.2. QKO1 Shows High In Vitro Immunogenicity

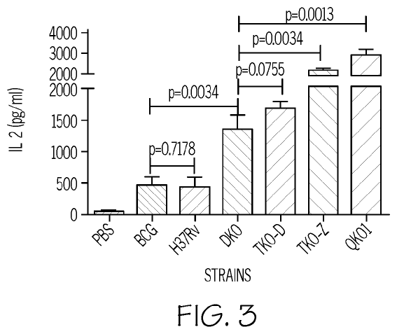

To determine whether the deletion of zmp1, dosR or both in DKO leads to increased immunogenicity, Applicants performed an in vitro antigen presentation assay based on the presentation of the antigenic epitope of Ag85B by APCs to T cells. In this method, mouse bone marrow derived macrophages (BMDMs) were infected with Mtb knockout strains separately and the infected macrophages were then cocultured with a T cell hybridoma specific for Ag85B₂₄₁₋₂₅₆ peptide for over a period of 16 h. The culture supernatants were then collected from the infected cells and assayed, via ELISA, for IL-2 levels released by the T cells in response to the presentation of Ag85B peptide. The results (FIG. 3) revealed that the T cells (BB7) cocultured with BMDMs infected with the TKO-Z and QKO1 strains released significantly higher levels of IL-2 in comparison to the DKO strain, indirectly indicating that BMDMs infected with these strains presented more antigenic epitopes to T cells. Particularly, the QKO1 strain exhibited two-fold increase in IL-2 levels as compared to DKO, suggesting that this vaccine strain is highly immunogenic. However, it should be noted that T cells cocultured with BMDM infected with the TKO-D strain also released higher levels of IL-2 but it is not statistically significant from that of DKO.

### Example 1.3. QKO1 is Processed Through PL Fusion

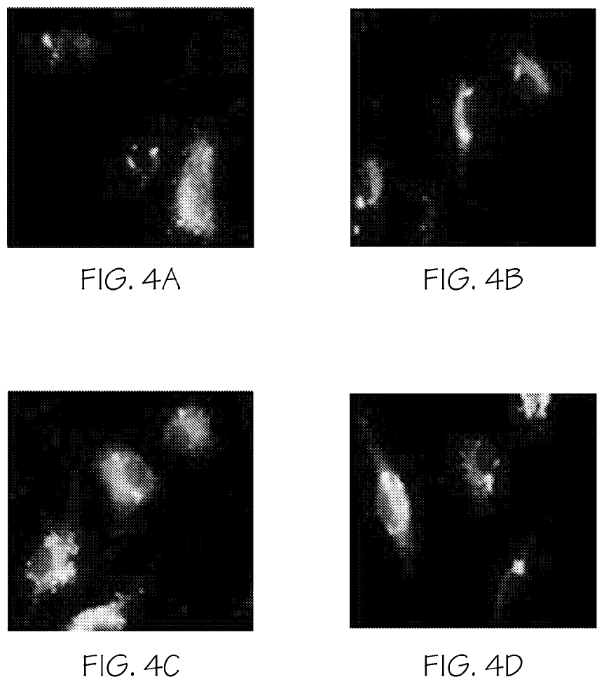

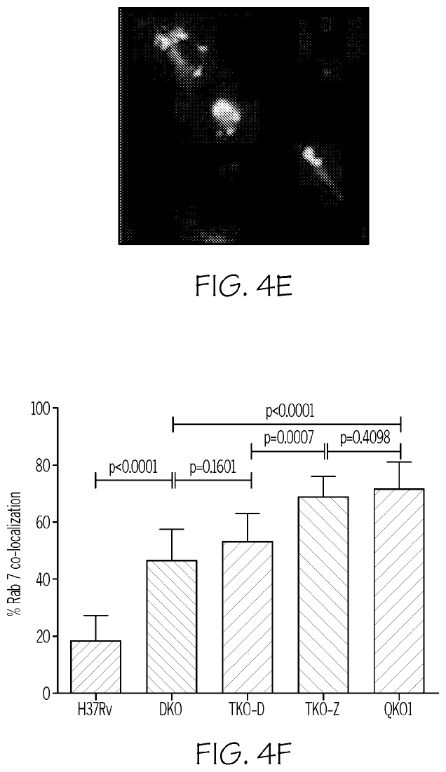

As discussed, immunogenicity could be due to processing and presentation of antigens through multiple pathways, although PL fusion pathway is the primary one. Earlier, Applicants have shown that the DKO infected macrophages exhibit enhanced PL fusion as compared to its parental strains. In order to determine whether additional deletion of genes in DKO leads to increased PL fusion, Applicants performed phagolysosome-bacterium colocalization experiments. Here, Applicants infected BMDMs with red fluorescent protein (RFP) expressing mutant Mtb strains (DKO, TKO-D, TKO-Z and QKO1) and control strain (H37Rv) and stained the infected cells with antibodies against Rab7, an early PL marker. The results revealed that, similar to that observed for antigen presentation experiments, TKO-Z and QKO1 strains colocalized with the phagolysosome at the highest levels, indicating that these two strains are efficiently processed through PL fusion (FIGS. 4A-4F). In addition to Rab7, the late PL marker Lamp1 also showed a similar colocalization patterns (data not shown) with the mutants, again reiterating that QKO1 is PL fusion competent.

### Example 1.4. QKO1 Induces Autophagy

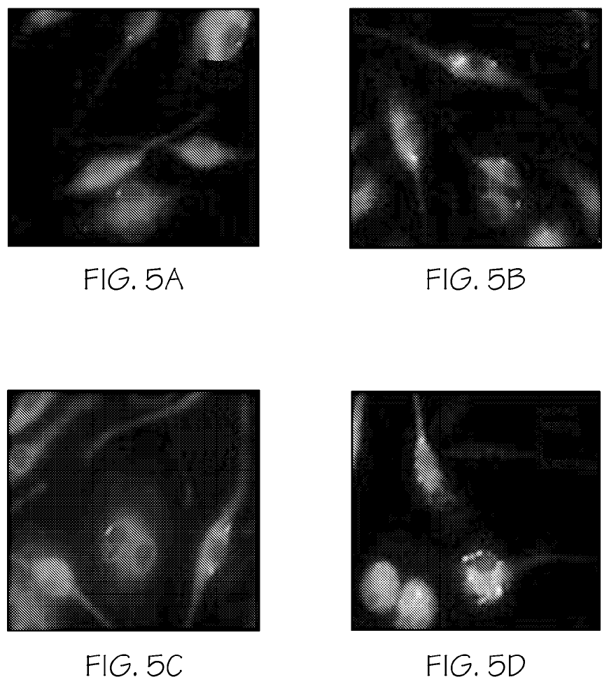

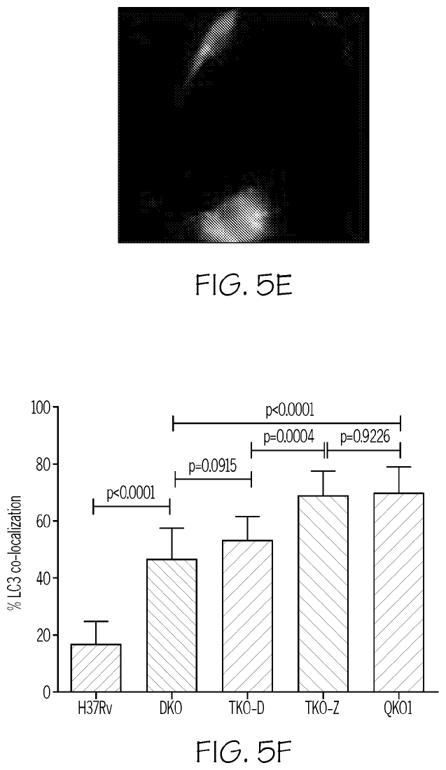

In mammalian cells, autophagy is a homeostatic mechanism which degrades the misfolded proteins and damaged organelles in the cytosol through autophago-lysosomes (APL). In recent years, it has been increasingly recognized that many intracellular pathogens are also internalized into the autophagosomes and delivered into APL for degradation, a process also known as xenophagy or macroautophagy. Previously, Applicants reported that BCG overexpressing Ag85B was processed through APL pathway. To understand whether Applicants' Mtb mutants induce autophagy in BMDMs, Applicants performed colocalization experiments as described for PL fusion in Example 1.3 but staining the phagosomes with autophagy marker LC3. Applicants found all Mtb mutants showed colocalization with APL but TKO-Z and QKO1, once again, showed the highest percentages of colocalization (FIGS. 5A-5F). These results indicate that TKO-Z and QKO1 are efficiently processed through APL pathway.

### Example 1.5. QKO1 Induces Apoptosis

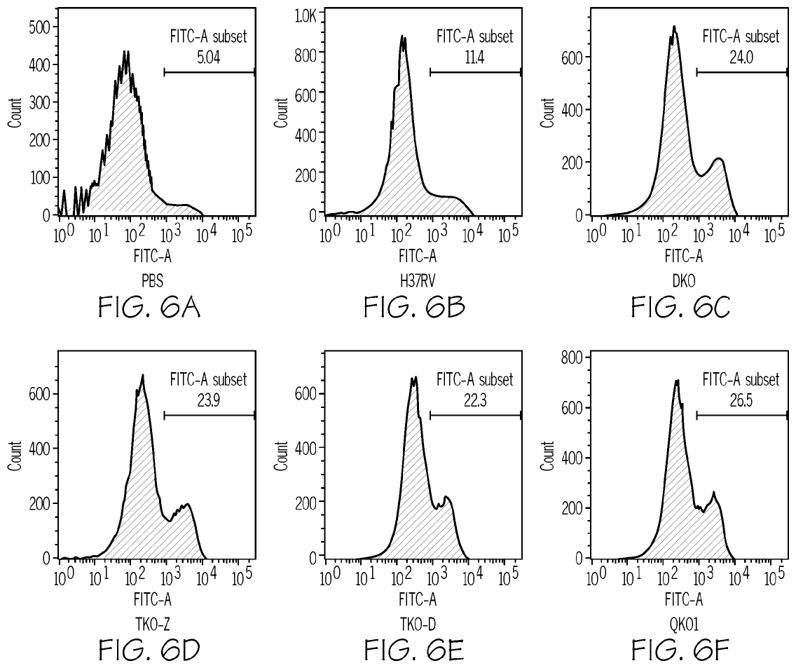

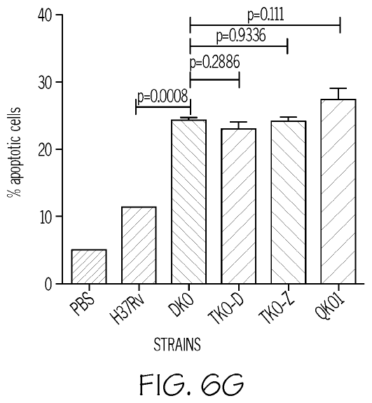

In addition to PL and APL pathways, apoptosis is also an alternate mechanism implicated in the processing and presentation of antigens because apoptotic bodies carry the infected bacteria. In general, Mtb is considered to have the ability to inhibit apoptosis of the infected cells. However, deletion of certain genes in Mtb has been shown to induce apoptotic death of macrophages. For example, Mtb nuoG gene which codes for a subunit of NADH dehydrogenase has been reported to induce autophagy in macrophages. Since the Mtb knockout mutants that Applicants generated have multiple gene deletions, Applicants aimed to understand whether these mutants could induce apoptosis in macrophages. Therefore, Applicants infected BMDMs with DKO, TKO-D, TKO-Z and QKO1 separately and assessed the cells for the induction of apoptosis by staining the cells with Annexin V. Flow cytometry of the infected cells revealed that all mutant strains including QKO1 induce significant apoptosis as compared to the wild type (H37Rv) control, although the mutant strains exhibit no significant differences between them (FIGS. 6A-6G). These results suggest the possibility that QKO1 vaccine can also be processed through the apoptotic pathway.

### Example 1.6. Inhibitors Confirm the Processing of QKO1 Antigens Through all Pathways

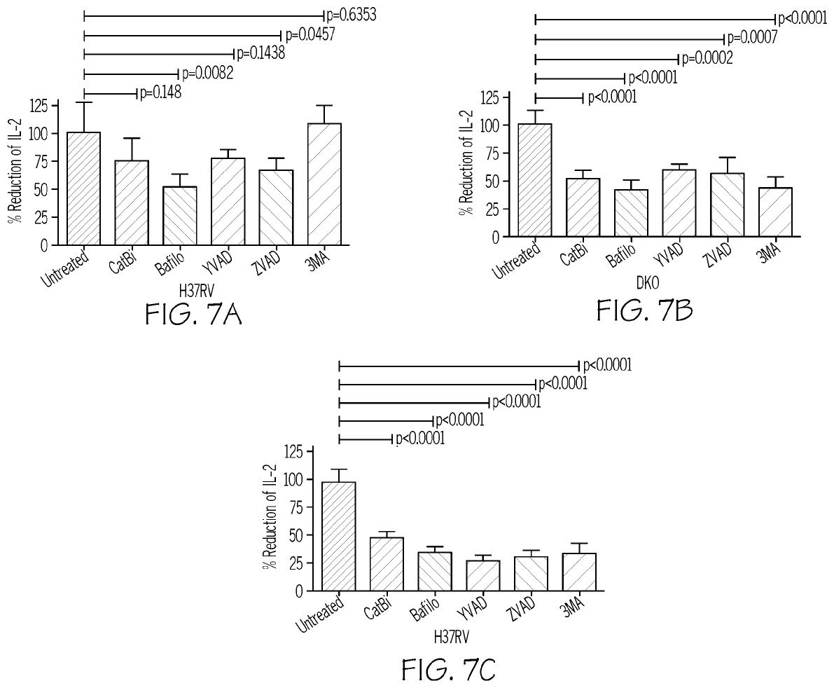

Although the colocalization studies and flow cytometry indicated that the QKO1 strain is processed through PL, APL and apoptotic pathways, Applicants sought to confirm whether processing through these pathways translates into presentation of antigens to T cells. To determine this, Applicants pretreated BMDMs with inhibitors for PL (Bafilomycin and cathepsin B), APL (3-methyl adenine) and apoptosis (z-VAD and y-VAD) pathways and then infected the cells with H37Rv, DKO and QKO1, separately. Afterwards, the antigen presentation (epitope of Ag85B) to T cells (BB7 hybridoma) by the infected BMDMs was determined. The results revealed that antigen presentation by QKO1 infected cells were inhibited by inhibitors of all pathways as in DKO infected cells (FIGS. 7A-7C). However, compared to DKO infected cells, APL and apoptosis inhibitors strongly inhibited the antigen presentation by QKO1 infected cells, indicating the robust involvement of these pathways in antigen presentation.

### Example 1.7. QKO1 Induces In Vivo Immunogenicity

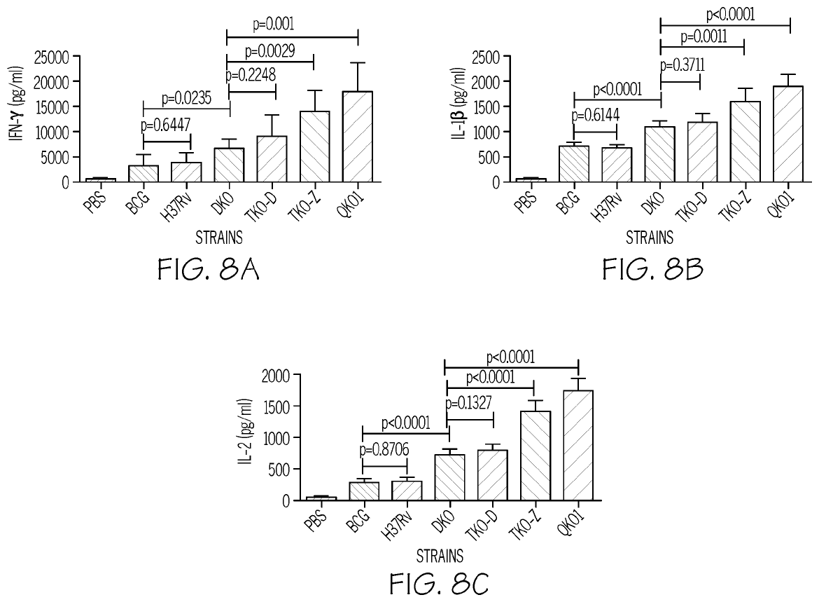

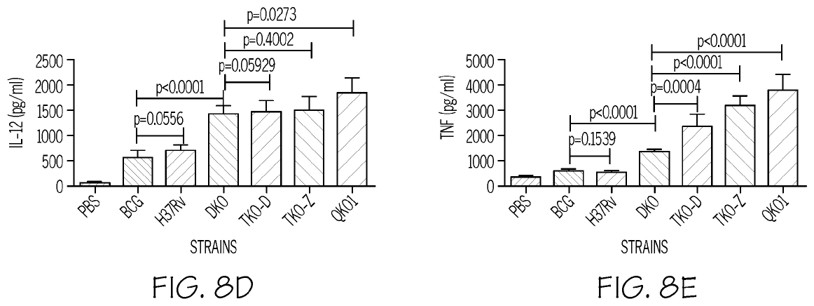

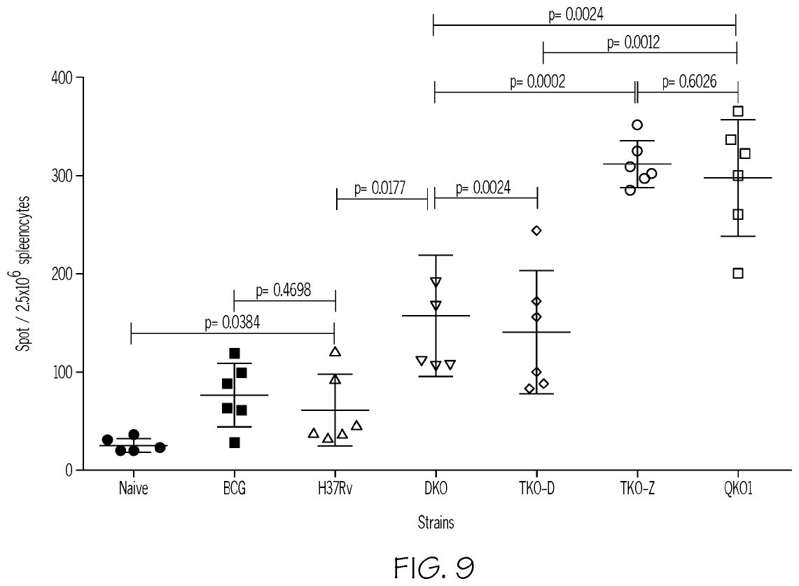

Since QKO1 induces strong immunogenicity in vitro, Applicants aimed to determine whether this phenomenon is also observed in vivo. Therefore, Applicants tested the immunogenicity of QKO1 strain and other Mtb strains in mice. Briefly, Applicants immunized C57BL/6 mice (both males and females (n=6)) with the vaccine strains (DKO, TKO-D, TKO-Z and QKO1) and control strains (BCG and H37RV) and after 30 days post-immunization Applicants collected the spleens. Splenocytes isolated from the spleens were later stimulated ex vivo with Mtb H37Rv Whole Cell Lysate (obtained from BEI Resources) and supernatants from these cultures were assayed for IFN-γ, IL-1β, IL-2 and TNF levels in ELISA. The results revealed that splenocytes of mice immunized with TKO-Z and QKO1 strains released the highest levels of antigen specific IFN-γ, IL-1β, IL-2, IL-12, and TNF cytokines (FIGS. 8A-8E). Splenocytes of immunized mice analyzed for ESAT6-CFP10 specific IFN-γ expressing cells in ELISPOT assay also revealed a similar trend (FIG. 9), indicating that QKO1 is highly immunogenic and a promising TB vaccine strain.

### Example 1.8. QKO1 is Severely Attenuated in Macrophages

Live bacterial vaccines should have an inherent attenuated ability to grow and multiply inside the host cells. Applicants have previously reported that the Mtb DKO (ΔfbpA-ΔsapM) strain was relatively attenuated in macrophages in comparison to its parental strains. Since QKO1 strain has two additional gene deletions, Applicants sought to know the ability of this strain to grow and survive within the intracellular environment. To accomplish this, Applicants infected BMDMs with Mtb mutant strains (DKO, TKO-D, TKO-Z and QKO1) and the control strain (H37Rv), and followed their viability over a period of time.

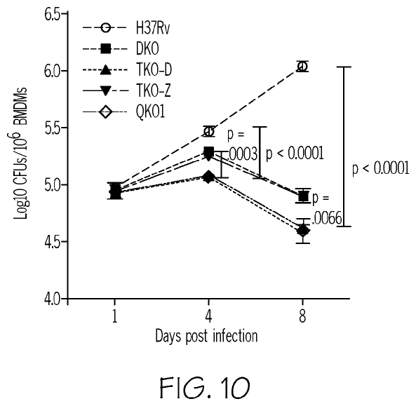

Data presented in FIG. 10, show that all mutant strains had a decline in survival within macrophages after 4 and 8 days of post-infection. Particularly the TKO-D and QKO1 strains had the greatest decreased ability to survive inside macrophages after 8 days of post-infection. In contrast, TKO-Z which has zmp1 deletion (but not dosR deletion) displayed a higher survival rate at this point. This suggested that the severely attenuated phenotype of QKO1 could be due to the deletion of dosR.

Overall, Applicants' results indicate that QKO1 is the most immunogenic strain among all the strains tested and, therefore, a promising vaccine candidate. Although TKO-Z shows properties similar to that of QKO1 in many respects, it is not as attenuated as QKO1 in the intracellular viability assay, indicating it still has some virulence properties.

### Example 2. Triple Knockout Bacterial Strains as a Vaccine Against Tuberculosis

In this Example, Applicants aim to improve BCG by rationally deleting genes. Applicants aim to sequentially delete three genes of BCG, namely sapM, zmp1 and nuoG, through homologous recombination, to result in a triple knockout BCG (BCG-TKO) strain. These genes encode proteins that enable BCG to evade host immune response by preventing phagosome-lysosomal fusion, autophagy, apoptosis and other related processes in the antigen presenting cells (APCs) such as dendritic cells and macrophages. The deletion of these genes could allow the BCG-TKO strain to be efficiently processed by APCs, which will lead to increased antigen presentation to the immune cells and enhanced in vivo immunogenicity and efficacy. Additionally, the deletion of these genes could reduce the virulence of the BCG, thus making the BCG-TKO to be HIV safe. Applicants plan to accomplish these goal with three aims: 1) construct a BCG-TKO strain through homologous recombination, 2) analyze the immunogenicity and safety of the BCG-TKO strain in a SCID mouse model, and 3) investigate the efficacy of the BCG-TKO in the regular and humanized mouse models with and without HIV infection.

Without further elaboration, it is believed that one skilled in the art can, using the description herein, utilize the present disclosure to its fullest extent. The embodiments described herein are to be construed as illustrative and not as constraining the remainder of the disclosure in any way whatsoever. While the embodiments have been shown and described, many variations and modifications thereof can be made by one skilled in the art without departing from the spirit and teachings of the invention. Accordingly, the scope of protection is not limited by the description set out above, but is only limited by the claims, including all equivalents of the subject matter of the claims. The disclosures of all patents, patent applications and publications cited herein are hereby incorporated herein by reference, to the extent that they provide procedural or other details consistent with and supplementary to those set forth herein.

What is claimed is:

1. A genetically altered bacterial strain, wherein the bacterial strain lacks functional versions of at least three of the following proteins:
FbpA;
SapM;
Zmp1;
DosR;
FadD26;
SigH;
nuoG; and
Eis.

2. The bacterial strain of claim 1, wherein the bacterial strain lacks functional versions of at least the following proteins:
FbpA;
SapM;
Zmp1; and
DosR.

3. The bacterial strain of claim 2, wherein the bacterial strain also lacks a functional version of the FadD26 protein.

4. The bacterial strain of claim 2, wherein the bacterial strain also lacks a functional version of the SigH protein.

5. The bacterial strain of claim 1, wherein the bacterial strain lacks functional versions of at least the following proteins:
FbpA;
SapM;
Zmp1;
DosR;
FadD26; and
SigH.

6. The bacterial strain of claim 1, wherein the bacterial strain lacks functional versions of at least the following proteins:
SapM;
Zmp1; and
nuoG.

7. The bacterial strain of claim 1, wherein the bacterial strain lacks functional versions of at least the following proteins:
SapM;
Zmp1; and
Eis.

8. The bacterial strain of claim 1, wherein the bacterial strain lacks functional versions of at least the following proteins:
SapM;
Zmp1;
Eis; and
nuoG.

9. The bacterial strain of claim 1, wherein the bacterial strain comprises a mutation or deletion of fbpA.

10. The bacterial strain of claim 1, wherein the bacterial strain comprises a mutation or deletion of sapM.

11. The bacterial strain of claim 1, wherein the bacterial strain comprises a mutation or deletion of zmp1.

12. The bacterial strain of claim 1, wherein the bacterial strain comprises a mutation or deletion of dosR.

13. The bacterial strain of claim 1, wherein the bacterial strain comprises a mutation or deletion of FadD26.

14. The bacterial strain of claim 1, wherein the bacterial strain comprises a mutation or deletion of SigH.

15. The bacterial strain of claim 1, wherein the bacterial strain comprises a mutation or deletion of nuoG.

16. The bacterial strain of claim 1, wherein the bacterial strain comprises a mutation or deletion of Eis.

17. The bacterial strain of claim 1, wherein the bacterial strain comprises a functional version of ESAT6.

18. The bacterial strain of claim 1, wherein the bacterial strain comprises a functional version of CFP10.

19. The bacterial strain of claim 1, wherein the bacterial strain is Mycobacterium tuberculosis (Mtb).

20. The bacterial strain of claim 1, wherein the bacterial strain is Mycobacterium bovis BCG (BCG).

21. The bacterial strain of claim 1, wherein the bacterial strain is suitable for use in treating or preventing a bacterial infection in a subject.

22. The bacterial strain of claim 1, wherein the bacterial strain is in a composition suitable for administration to a subject.

23. The bacterial strain of claim 22, wherein the composition is in the form of a vaccine.

24. A method of treating or preventing a bacterial infection in a subject, said method comprising:
administering to the subject a bacterial strain, wherein the bacterial strain lacks functional versions of at least three of the following proteins:
FbpA;
SapM;
Zmp1;
DosR;
FadD26;
SigH;
nuoG; and
Eis.

25. The method of claim 24, wherein the bacterial strain lacks functional versions of at least the following proteins:
FbpA;
SapM;
Zmp1; and
DosR.

26. The method of claim 24, wherein the bacterial strain also lacks a functional version of the FadD26 protein.

27. The method of claim 24, wherein the bacterial strain also lacks a functional version of the SigH protein.

28. The method of claim 24, wherein the bacterial strain lacks functional versions of at least the following proteins:
FbpA;
SapM;
Zmp1;
DosR;
FadD26; and
SigH.

29. The method of claim 24, wherein the bacterial strain lacks functional versions of at least the following proteins:
SapM;
Zmp1; and
nuoG.

30. The method of claim 24, wherein the bacterial strain lacks functional versions of at least the following proteins:
SapM;
Zmp1; and
Eis.

31. The method of claim 24, wherein the bacterial strain lacks functional versions of at least the following proteins:
SapM;
Zmp1;
Eis; and
nuoG.

32. The method of claim 24, wherein the bacterial strain comprises a functional version of ESAT6.

33. The method of claim 24, wherein the bacterial strain comprises a functional version of CFP10.

34. The method of claim 24, wherein the administering occurs by a method selected from the group consisting of intravenous administration, subcutaneous administration, transdermal administration, topical administration, intraarterial administration, intrathecal administration, intracranial administration, intraperitoneal administration, intraspinal administration, intranasal administration, intraocular administration, oral administration, intratumor administration, and combinations thereof.

35. The method of claim 24, wherein the bacterial infection is tuberculosis.

36. The method of claim 24, wherein the subject is a human being.

37. The method of claim 24, wherein the administering prevents the bacterial infection.

38. The method of claim 24, wherein the administering treats the bacterial infection.

39. The method of claim 24, wherein the administering elicits an enhanced immune response against the bacterial infection in the subject.

40. The method of claim 39, wherein the enhanced immune response is characterized by enhanced phagolysosomal processing of the bacterial strain by antigen presenting cells.

41. The method of claim 39, wherein the enhanced immune response is characterized by enhanced IL-2 production in the subject.
